(19) United States
(12) Patent Application Publication
(10) Pub. No.: US 2016/0100887 A1
(43) Pub. Date: Apr. 14, 2016

(54) CASE STRUCTURE FOR ELECTRONIC DEVICE

(71) Applicant: GiMer Medical Co., Ltd., New Taipei City (TW)

(72) Inventors: Chen-Tun WU, New Taipei City (TW); Chan-Yi CHENG, New Taipei City (TW)

(21) Appl. No.: 14/873,051

(22) Filed: Oct. 1, 2015

(30) Foreign Application Priority Data
Oct. 1, 2014 (TW) 103217435

(51) Int. Cl. A61B 19/00; H02B 1/32

(52) U.S. Cl. CPC A61B 19/02

(57) ABSTRACT

A housing structure for covering electronic device comprises a housing. The housing comprises a first housing portion and a second housing portion. The first housing portion and the second housing portion are sealed together, and the material of the first housing portion and the second housing portion is polyether ether ketone.

### CROSS REFERENCE TO RELATED APPLICATIONS

This Non-provisional application claims priority under 35 U.S.C. §119(a) on Patent Application No(s). 103217435 filed in Taiwan, Republic of China on Oct. 1, 2014, the entire contents of which are hereby incorporated by reference.

### BACKGROUND

1. Technical Field

The invention relates to a housing structure for electronic device, in particular to a housing structure implanted in organism which is waterproof and prevents the transmission line from loosing.

2. Related Art

As electronic technique develops, electronic devices have been applied to various fields for example implantable medical electronic device. To guarantee safety and life time of the implantable medical electronic device, waterproof is a major requirement for such device. Regarding waterproofing, damp-proofing and so on, components of electronic device such as connection socket, housing through hole or others need tightly fabricating to avoid damp and oxidization problems of electronic components in the electronic device caused by the damp seeping through hole of the electronic device. Seriously, the short circuit due to seeping damp may damage the circuit board. Therefore, it is necessary for the implantable medical electronic device to meet strict waterproof requirements to avoid endangering the patient.

To solve waterproof and damp-proof problems, a waterproof rubber ring is additionally utilized in the conventional electronic device to close the gap between the connector and the device body for avoiding damp. However, taking the implantable medical electronic device for example, the overall outer diameter of the electrode lead of the implantable medical electronic device needs to be smaller than the inner diameter of the employed needle guide due to the requirements for minimally invasive implantation surgery. Thus, limited to the tiny electrode lead, an ordinary waterproof rubber ring disposed on the electrode lead is not sufficiently watertight if no auxiliary element compresses the waterproof rubber ring. As a result, damp may still seep through the gap between the electrode lead and the connector into the inside of the implantable medical electronic device. The electronic components in the implantable medical electronic device may be damped and oxidized, or the circuit in the implantable medical electronic device may short-circuit and be damaged, which is dangerous to the patient.

Therefore, a housing for hermetically covering an implantable medical electronic device of which the material is not harmful to the human body when implanted in the human body is needed.

### SUMMARY

An aspect is to provide a housing structure for electronic device of which the material is PEEK (polyether ether ketone) for low danger of implantation of electronic device into human body.

A housing structure for covering electronic device comprises a housing.

The housing comprises a first housing portion and a second housing portion. The first housing portion and the second housing portion are sealed together, and the material of the first housing portion and the second housing portion is polyether ether ketone.

In summary, the housing structure for medical electronic device combines a first housing portion and a second housing portion, and the material of the first housing portion and the second housing portion is PEEK. Because such material is resistant to heat, corrosion, hydrolysis and radiation, etc. Thus, the material of the housing is not harmful to the human body, so the electronic device implanted in the human body may be as little as possible dangerous to the human body.

### BRIEF DESCRIPTION OF THE DRAWINGS

The embodiments will become more fully understood from the detailed description and accompanying drawings, which are given for illustration only, and thus are not limitative of the present invention, and wherein:

FIG. 1 is a schematic diagram of the appearance of a housing structure according to an embodiment;

FIG. 2 is an exploded view of the housing structure in FIG. 1;

FIG. 3 is an exploded view of the connector of the housing structure in FIG. 1;

FIG. 4 and FIG. 5 are sectional diagrams of the housing structure in FIG. 1;

FIG. 6 is a schematic diagram of the appearance of the housing structure according to another embodiment; and

FIG. 7 is an exploded view of the housing structure in FIG. 6.

### DETAILED DESCRIPTION OF THE INVENTION

The embodiments of the invention will be apparent from the following detailed description, which proceeds with reference to the accompanying drawings, wherein the same references relate to the same elements.

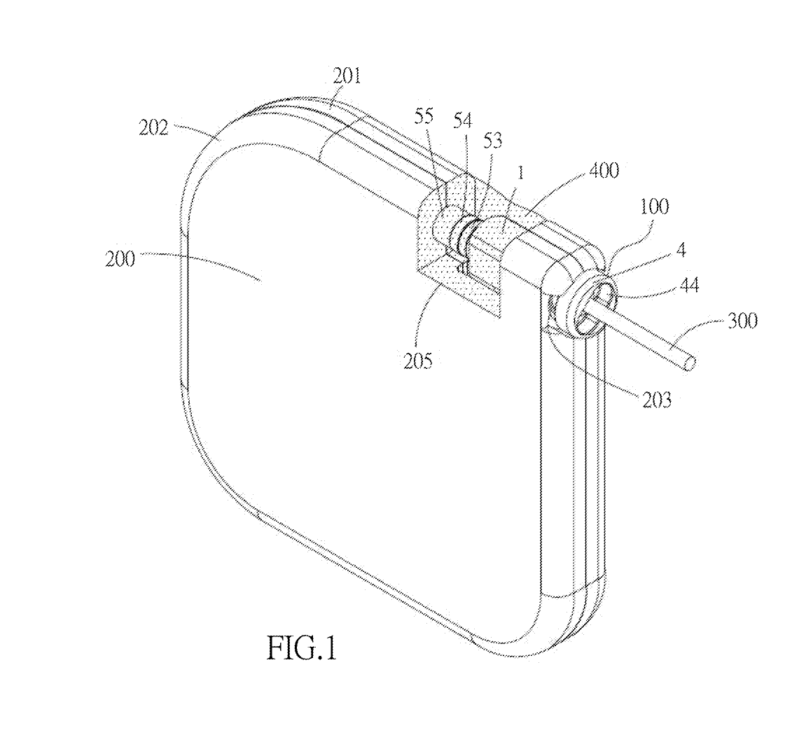

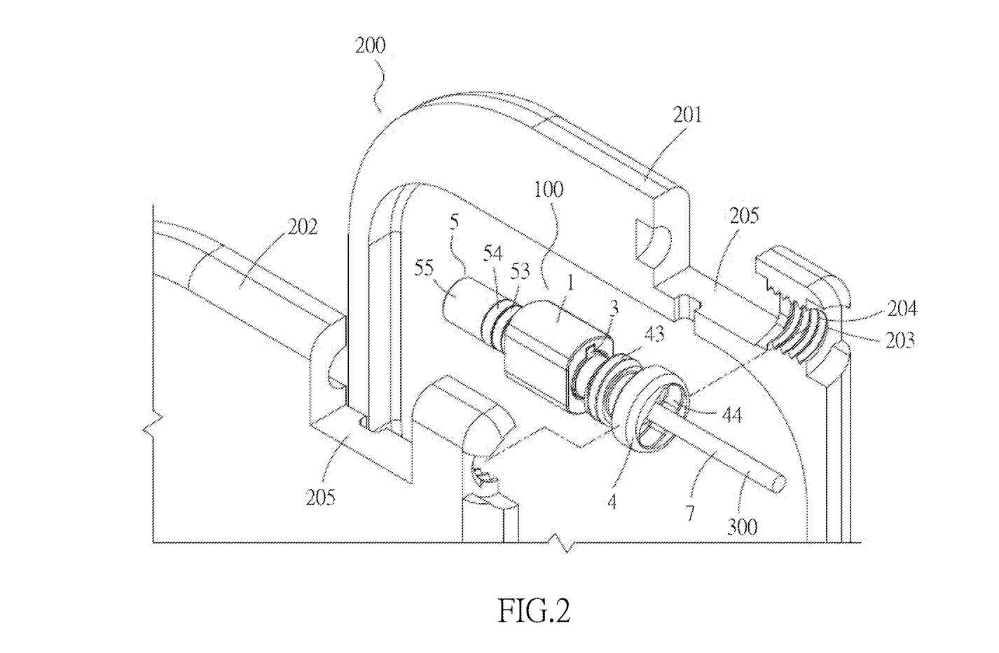

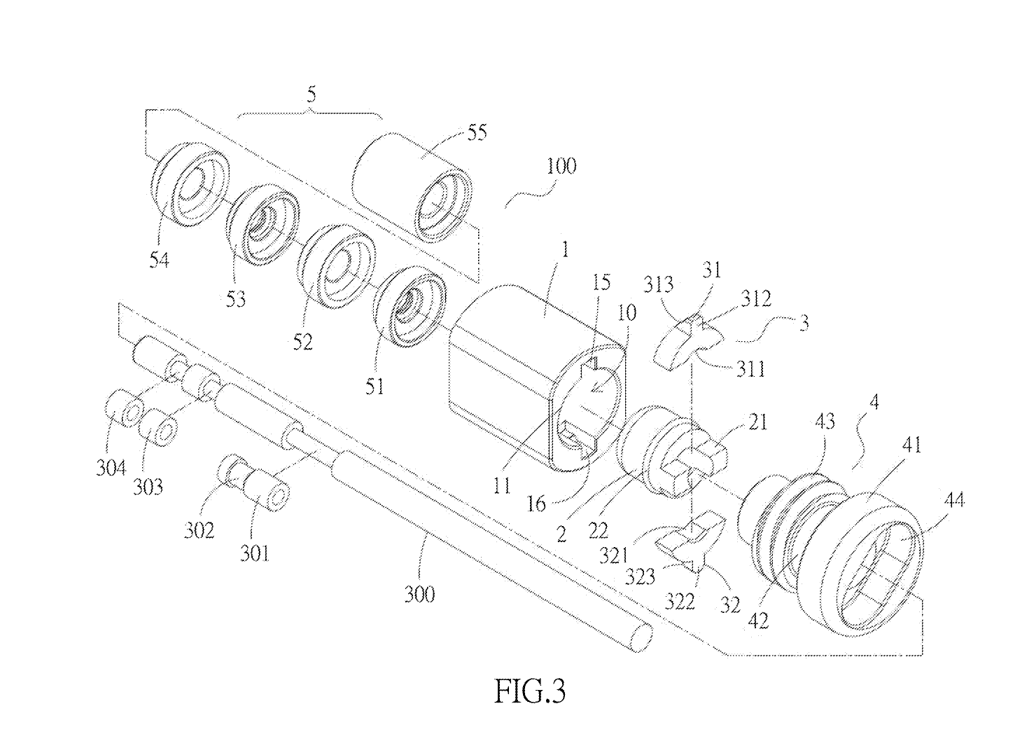

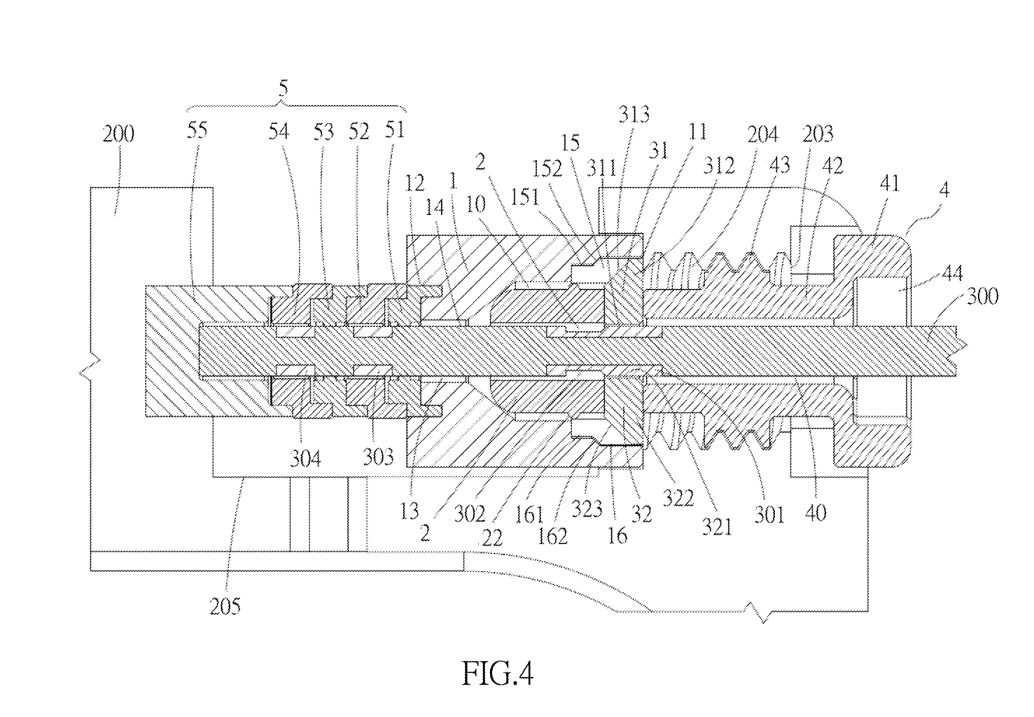

Referring to FIG. 1 to FIG. 5, the disclosure illustrates a housing structure D, in particular to the housing structure D which is adapted to cover and carry an electronic device for implantation into an individual. The housing structure D comprises a housing 200, a connector 100, a transmission line 300 and a seal 400.

In the disclosure, the individual preferably is an organism, and it may include mammals such as mouse, human, rabbit, cattle, sheep, pig, monkey, dog, cat, etc. Preferably, it is human.

The housing 200 comprises a first housing portion 201 and a second housing portion 202. The first housing portion 201 and the second housing portion 202 are sealed together. In the embodiment, the material of the first housing portion 201 and the second housing portion 202 is PEEK (polyether ether ketone), so the housing 200 can be resistant to heat, corrosion, hydrolysis and radiation, etc. Thus, the material of the housing is not harmful to the human body, so the electronic device implanted in the human body may be as little as possible dangerous to the human body.

The housing 200 has an opening portion 203 at its one side. The opening portion 203 has a first fastening portion 204 on its inner wall. The housing 200 has a hollow portion 205 at the other side of the opening portion 203. The hollow portion 205 and its surroundings may be filled with a seal 400 (e.g. resin or plastic).

In the embodiment, the first housing portion 201 and the second housing portion 202 are sealed together by ultrasonic welding, so the housing 200 can completely hermetically cover and carry an electronic device.

The connector 100 is sealed in the opening portion 203 of the housing 200. The connector 100 comprises a base 1, a sleeve 2, a clamping unit 3, a fastener 4 and a conductive module 5.

The base 1 and the housing 200 detachably combine together. Under a complete combination, relatively, the base 1 located at the hollow portion 205. In other words, the base 1 is disposed in the hollow portion 205. The base 1 has a first opening 11 at its one end, and has a second opening 12 at the other end. The first opening 11 and the opening portion 203 of the housing 200 are disposed correspondingly. The base 1 comprises a cavity 10 at its inner. The cavity 10 communicates with the first opening 11, and communicates with the second opening 12 through a passage 13. The cavity 10 comprises a narrowing portion 14 near the passage 13. The shape of the narrowing portion 14 is not limited, and in the embodiment, it is cone-shaped for example. The narrowing portion 14 gradually narrows toward the second opening 12, and gradually widens toward the first opening 11.

Besides, the cavity 10 has a first guide 15 and a second guide 16 near the first opening 11. The first guide 15 has a first stopper 151 at the junction of the first guide and the cavity 10. The first stopper 151 has a first slanted surface 152 toward the first opening 11. The second guide 16 has a second stopper 161 at the junction of the second guide 16 and the cavity 10. The second stopper 161 has a second slanted surface 162 toward the first opening 11.

The sleeve 2 is disposed in the cavity 10. The sleeve 2 comprises a first through hole 20. One end of the sleeve 2 contacts the narrowing portion 14, and two corresponding first protrusions 21 protrude at the other end. The sleeve 2 has at least one second protrusion 22 enclosing its external surface, and the second protrusion 22 tightly contacts the cavity 10. In the embodiment, the sleeve 2 is an elastomer, and its material is for example silicone or PU (Polyurethane). Thus, the sleeve 2 is compressed by the narrowing portion 14 of the base 1 to deform, and disposed within the narrowing portion 14.

Referring to FIG. 1 to FIG. 5, the clamping unit 3 is similarly disposed in the cavity 10 of the base 1. It comprises a first clamping member 31 and a second clamping member 32. The first clamping member 31 and the second clamping member 32 are disposed in the cavity 10 of the base 1 and contact the sleeve 2. For example, the first clamping member 31 is semi-circle-shaped, and it has a first indentation 311 caving at its one side and has a third protrusion 312 at the other side. The third protrusion 312 is disposed at the first guide 15 of the base 1. After they combine together, the third protrusion 312 has a third slanted surface 313 against the first stopper 151, and the third slanted surface 313 matches the first slanted surface 152.

In the embodiment, the second clamping member 32 is semi-circle-shaped, and it has a second indentation 321 caving at one side and has a fourth protrusion 322 at the other side. After they combine together, the second indentation 321 and the first indentation 311 are disposed correspondingly. The fourth protrusion 322 is located at the second guide 16 of the base 1, and the fourth protrusion 322 has a fourth slanted surface 323 against the second stopper 161. The fourth slanted surface 323 matches the second slanted surface 162. The first clamping member 31 and the second clamping member 32 are respectively located at two sides of the first protrusion 21 of the sleeve 2. The first indentation 311 of the first clamping member 31 and the second indentation 321 of the second clamping member 32 are disposed against the third indentation 302 of the orientation member 301 of the transmission line 300.

In the embodiment, the fastener 4 and the opening portion 203 of the housing 200 combine together. The fastener 4 comprises a base 41. The base 41 extends at its one end to form an extension portion 42. The extension portion 42 can contact the clamping unit 3. The fastener 4 has a second fastening portion 43 on the external surface of the extension portion 42. The second fastening portion 43 and the first fastening portion 204 of the housing 200 combine together. In addition, the fastener 4 further comprises a second through hole 40 passing through the base 41 and the extension portion 42. Moreover, the fastener 4 has the slot 44 at the other end of the base 41.

In the embodiment, the first fastening portion 204 and the second fastening portion 43 respectively have corresponding screw structures to match together, so the first fastening portion 204 and the second fastening portion 43 can interlock, and the fastener 4 is able to linearly (straight) move and perform positioning. For example without limitation to, the first fastening portion 204 may be a slot (or protrusion), and the second fastening portion 43 may be a protrusion (or slot) corresponding to the structure of the first fastening portion 204. By the engagement of the first fastening portion 204 and the second fastening portion 43, similarly, the fastener 4 is also able to linearly (straight) move and perform positioning. Moreover, the slot 44 also allow a placement of a designate external object to rotate the fastener 4.

The conductive module 5 is detachably disposed in the second opening 12 of the base 1. After they combine together, similarly, the conductive module 5 is located at the hollow portion 205. The conductive module 5 comprises a first ring 51, a first conductive ring 52, a second ring 53, a second conductive ring 54 and a sealing member 55. The first ring 51, the first conductive ring 52, the second ring 53, the second conductive ring 54 and the sealing member 55 combine in sequence. The first ring 51 is detachably disposed in the second opening 12 of the base 1.

The transmission line 300 comprises an orientation member 301, and the orientation member 301 comprises a third indentation 302. The transmission line 300 further comprises a first conductive member 303 and a second conductive member 304. The transmission line 300 may pass through the second through hole 40 of the fastener 4, the clamping unit 3, the first through hole 20 of the sleeve 2, the cavity 10 and the passage 13 of the base 1 in sequence to extend to the exterior of the base 1. The transmission line 300 extending to the exterior of the base 1 may further pass through the conductive module 5. The transmission line 300 is sealed and covered with the base 1 and the conductive module 5. After they combine together, the first conductive member 303 of the transmission line 300 electrically contacts the first conductive ring 52 of the conductive module 5, and the second conductive member 304 of the transmission line 300 electrically contacts the second conductive ring 54 of the conductive module 5.

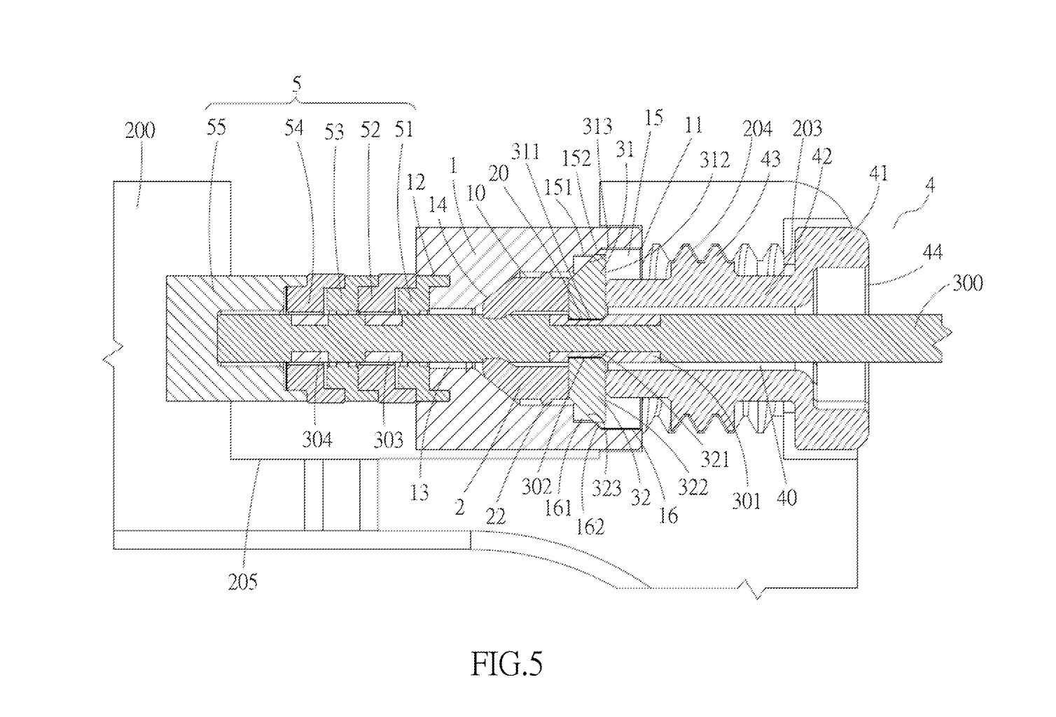

The fastener 4 linearly moves to compress the clamping unit 3 and the sleeve 2. When the sleeve 2 compresses toward the second opening 12 (shown in FIG. 5), one end of the sleeve 2 is compressed to deform by the narrowing portion 14 of the base 1, so the sleeve 2, the inner wall of the cavity 10 of the base 1 and the transmission line 300 tightly combine together to waterproof and seal the base 1. Moreover, the second protrusion 22 on the external surface of the sleeve 2 tightly contacts the cavity 10 of the base 1 to further waterproof the connector 100. Thus, external damp is prevented from seeping through the opening portion 203 of the housing 200 and the base 1 into the housing 200, which avoids damage to the circuits within the electronic device due to short circuit.

The fastener 4 linearly moves to compress the clamping unit 3. When the clamping unit 3 moves toward the second opening 12, the third protrusion 312 and the third slanted surface 313 of the first clamping member 31 respectively contact the first stopper 151 and the first slanted surface 152, and the fourth protrusion 322 and the fourth slanted surface 323 of the second clamping member 32 respectively contact the second stopper 161 and the second slanted surface 162. Therefore, the first clamping member 31 and the second clamping member 32 move toward the transmission line 300 and gradually become narrow. At the moment, the first indentation 311 of the first clamping member 31 and the second indentation 321 of the second clamping member 32 respectively can correspondingly clamp the third indentation 302 of the orientation member 301 of the transmission line 300 (shown in FIG. 5). Thus, the positioning is stable when the transmission line 300 passes through the base 1, and the transmission line 300 is prevented from being loosened from the base 1. The clamping unit 3 linearly moves to perform positioning. When the outmost surface of the third protrusion 312 of the first clamping member 31 is aligned with the surface of the first stopper 151 and the outmost surface of the fourth protrusion 322 of the second clamping member 32 is aligned with the surface of the second stopper 161, the first clamping member 31 and the second clamping member 32 of the clamping unit 3 stop moving toward the transmission line 300 and becoming narrow if the fastener 4 continues linearly moving or to move to compress the clamping unit 3. Thus, the first clamping member 31 and the second clamping member 32 are prevented from excessively compressing the transmission line 300 to avoid damage to the transmission line 300.

For example, the hollow portion 205 of the housing 200 may be filled with a seal 400. As the base 1 of the connector 100 and the conductive module 5 are sealed and covered with the seal 400, the damp is prevented from seeping into the housing.

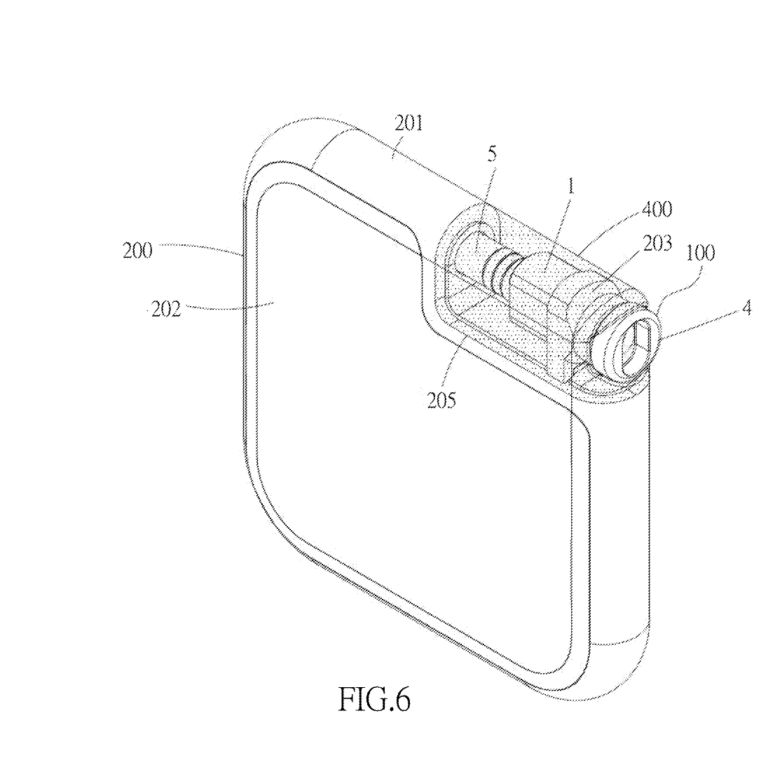

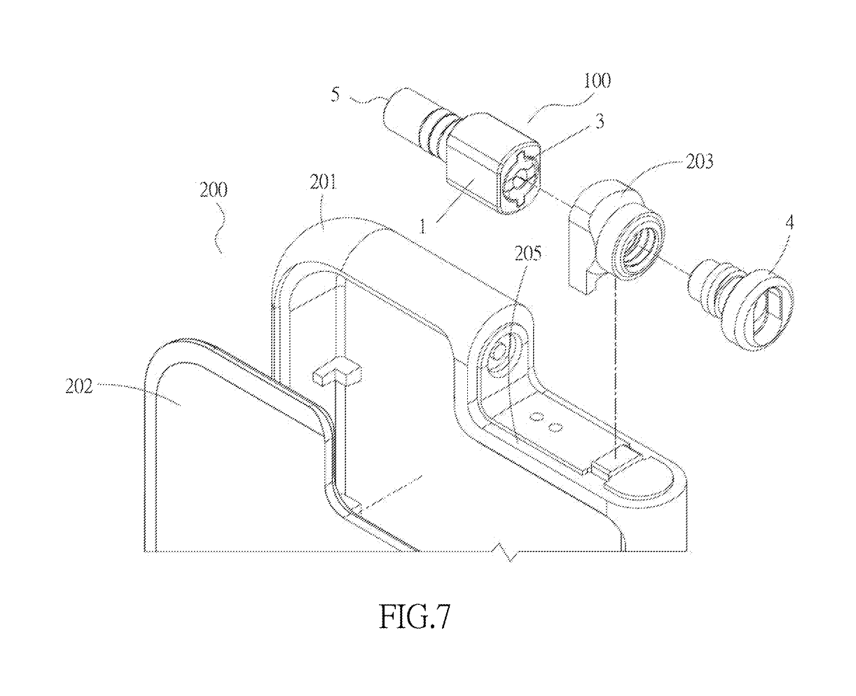

Referring to FIG. 6 and FIG. 7, in the embodiment, the housing 200 and the opening portion 203 of the housing 200 are integratively formed together (shown in FIG. 1), namely, the opening portion 203 and the housing 200 are a single component. Alternatively, referring to FIG. 7, the opening portion 203 is an independent component and it and the housing 200 are not integratively formed. In the embodiment, the opening portion 203 is adjacent to one side of the hollow portion 205 of the housing 200. Similarly, the opening portion 203 has a first fastening portion 204 on its inner wall. If the opening portion 203 is an independent component from the housing 200, it is more durable. Because the descriptions about that the connector 100 is sealed in the opening portion 203 and that the outside of the opening portion 203 and the hollow portion 205 are filled with the seal 400 for sealing are similar to the previous embodiments, they are not repeated here. In summary, the housing structure for medical electronic device combines a first housing portion and a second housing portion, and the material of the first housing portion and the second housing portion is PEEK. Because such material is resistant to heat, corrosion, hydrolysis and radiation, etc. Thus, the material of the housing is not harmful to the human body, so the electronic device implanted in the human body may be as little as possible dangerous to the human body.

Although the invention has been described with reference to specific embodiments, this description is not meant to be construed in a limiting sense. Various modifications of the disclosed embodiments, as well as alternative embodiments, will be apparent to persons skilled in the art. It is, therefore, contemplated that the appended claims will cover all modifications that fall within the true scope of the invention.

What is claimed is:

1. A housing structure for covering electronic device, comprising:
a housing comprising a first housing portion and a second housing portion, wherein the first housing portion and the second housing portion are sealed together, the material of the first housing portion and the second housing portion is polyether ether ketone.

2. The housing structure of claim 1, wherein the first housing portion and the second housing portion are sealed together by ultrasonic welding.

3. The housing structure of claim 1, wherein the housing comprises an opening portion, and the opening portion comprises a first fastening portion on an inner wall of the opening portion.

4. The housing structure of claim 1, further comprising an opening portion, wherein the opening portion comprises a first fastening portion on an inner wall of the opening portion.

5. The housing structure of claim 3, further comprising:
a connector sealed in the opening portion of the housing, wherein the connector comprises a fastener, the fastener comprises a second fastening portion, and the second fastening portion and the first fastening portion of the housing detachably combine together.

6. The housing structure of claim 4, further comprising:
a connector sealed in the opening portion of the housing, wherein the connector comprises a fastener, the fastener comprises a second fastening portion, and
the second fastening portion and the first fastening portion of the housing detachably combine together.

7. The housing structure of claim 5, wherein the connector further comprising:
a base wherein the base and the housing detachably combines together, a first opening is located at one end of the base and a second opening is located at the other end of the base, the first opening and the opening portion of the housing are disposed correspondingly, the base comprises a cavity, the cavity communicates with the first opening and communicates with the second opening through a passage, the cavity comprises a narrowing portion near the passage, the cavity comprises a first guide and a second guide near the first opening, the first guide comprises a first stopper at the junction of the first guide and the cavity, and the second guide comprises a second stopper at the junction of the second guide and the cavity;
a sleeve disposed in the cavity and comprising a first through hole, wherein one end of the sleeve contacts the narrowing portion and is compressed to deform by the narrowing portion; and
a clamping unit comprising a first clamping member and a second clamping member, wherein the first clamping member and the second clamping member are disposed in the cavity of the base and contact the sleeve, the first clamping member comprises a first indentation caving at its one side and comprises a third protrusion at the other side, the third protrusion detachably engages the first guide, the third protrusion comprises a third slanted surface against the first stopper, the second clamping member comprises a second indentation caving at its one side and comprises a fourth protrusion at the other side, the second indentation and the first indentation are disposed correspondingly, the fourth protrusion detachably engages the second guide, and the fourth protrusion comprises a fourth slanted surface against the second stopper.

8. The housing structure of claim 5, wherein the fastener comprises a base, the base extends at its one end to form an extension portion, the fastener comprises a second through hole passing through the base and the extension portion, the second fastening portion is disposed on the extension portion, the extension portion contacts the clamping unit, and the fastener linearly moves to compress the clamping unit and the sleeve.

9. The housing structure of claim 7, further comprising:
a transmission line sequentially passing through the second through hole of the fastener, the clamping unit, the first through hole of the sleeve, and the cavity and the passage of the base to extend to the exterior of the base, the transmission line comprises an orientation member, the orientation member comprises a third indentation, the first indentation of the first clamping member and the second indentation of the second clamping member clamp the third indentation of the orientation member.

10. The housing structure of claim 7, wherein the sleeve is an elastomer, the sleeve comprises at least one second protrusion enclosing the external surface of the sleeve, and the second protrusion tightly contacts the cavity of the base.

11. The housing structure of claim 7, wherein the narrowing portion is cone-shaped, and the narrowing portion gradually narrows toward the second opening and gradually widens toward the first opening.

12. The housing structure of claim 7, wherein the first stopper comprises a first slanted surface toward the first opening, the second stopper comprises a second slanted surface toward the first opening, the first slanted surface matches the third slanted surface of the first clamping member, and the second slanted surface matches the fourth slanted surface of the second clamping member.

13. The housing structure of claim 6, further comprising:
a conductive module detachably disposed in the second opening of the base and comprising a first ring, a first conductive ring, a second ring, a second conductive ring and a sealing member, wherein the first ring, the first conductive ring, the second ring, the second conductive ring and the sealing member combine in sequence, the first ring is detachably disposed in the second opening of the base, the transmission line extends to exterior of the base and passes through the conductive module, the transmission line comprises a first conductive member and a second conductive member, the first conductive member electrically contacts the first conductive ring, and the second conductive member electrically contacts the second conductive ring.

14. The housing structure of claim 7, wherein the housing comprises a hollow portion, the base and the conductive module are located in the hollow portion, a seal is disposed in the hollow portion of the housing, and the base and the conductive module are sealed and covered with the seal.

15. The housing structure of claim 14, wherein the material of the seal is resin or plastic.

16. The housing structure of claim 14, wherein the opening portion is an independent component, the opening portion is adjacent to the hollow portion, and the seal seals the outside of the opening portion and the hollow portion.

17. The housing structure of claim 7, wherein the opening portion and the housing are a single component.

18. The housing structure of claim 1, wherein the housing structure is a housing of an implantable electronic device for human body.
